(19) United States
(12) Patent Application Publication
(10) Pub. No.: US 2021/0060206 A1
(43) Pub. Date: Mar. 4, 2021

(54) Tilapia Dried Tissue – Revi-Aid

(71) Applicant: John Chung Nguyen, Reseda, CA (US)

(72) Inventor: John Chung Nguyen, Reseda, CA (US)

(21) Appl. No.: 16/558,085

(22) Filed: Aug. 31, 2019

(51) Int. Cl. A61L 27/36; A61K 35/60; A61K 38/39; A61K 9/70; A61L 27/24; A61L 27/60; A61L 27/54; A61F 13/02

(52) U.S. Cl. CPC A61L 27/362; A61K 35/60; A61K 38/39; A61K 9/7007; A61L 27/3687; A61L 27/60; A61L 27/54; A61F 13/0206; A61L 27/24

(57) ABSTRACT

The invention is employing the application of tilapia skin that has been processed and preserved in a dried tissue stage. To reach dried tissue stage, the fish skin is required to undergo treatment of Gludehyde and Glycerin solutions that is performed inside the clean room. The skin in new dried stage is then integrated with an adhesive strip and sterile gauze inside the clean room to create a new type of Ready-To-Use bandage that can be utilized as medical dressing for skin injuries, namely Revi-Aid.

The skin of the tilapia is harvested after the fish is mutilated. The skin is washed and cleaned with running cold water to remove all impurities such as excess muscles, blood, and extra fiber. The fish skin is then transferred to the Clean Room to process using the following method:

Step 1: Place the skin in a container and wash with sterile saline.

Step 2: Place the skin in a sterile container containing 0.25% Gludehyde solution.

Step 3: Place the skin in a container with 20% alcohol solution for 24 hours to sterilize.

Step 4: Wash the skin with sterile saline once more.

Step 5: Place the skin in a container with 30% Glycerin solution for calibrating time.

Step 6: Remove the skin from the container and hang to dry for 72 hours with room temperature inside the Clean Room.

Step 7: The skin is now in dried tissue stage. The skin can now be collected and cut into set sizes and place inside the bandage.

Step 8: Place the bandage inside a vacuum-sealed bag. The final product is now a Ready-To-Use fish skin bandage, namely Revi-Aid.

Step 9: When needing to use Revi-Aid, remove it from the sealed vacuum package by tear along the guiding tear line (FIG. 3). The Revi-Aid will then be pulled out of the packaging. Users will continue pulling the tab from right to left (FIG. 3) to expose the dried tissue fish skin inside the Revi-Aid. Now, to apply the dried tissue fish skin side of the Revi-Aid on the surface of the skin injuries accordingly

### TECHNICAL

The invention is employing the application of tilapia skin that has been processed and preserved in a dried tissue stage. To reach dried tissue stage, the fish skin is required to undergo treatment of Gludehyde and Glycerin solutions that is performed inside the clean room. The skin in new dried stage is then integrated with an adhesive strip and gauze inside the clean room to create a new type of Ready-To-Use Bandage that can be utilized as medical dressing for skin injuries, namely Revi-Aid. The Revi-Aid contains the skin at dried tissue stage that has been sterile, treated, and packed inside the clean room, hence, the skin can be Ready-To-Use at convenience without going through the rigorous process of additional steps of cleaning and preparing the skin prior to use.

### BRIEF SUMMARY

An innovative breakthrough with dried tissue technology using fish skin will transform how burned victims are treated. Currently, burned victims undergo a painful process of harvesting healthy skin from other parts of the body to cover the burned area. With this new dried tissue fish skin, burned victims will no longer required to go through the excruciating painful procedure to heal. This dried tissue fish skin can be stored as dried material; hence can be implemented directly into bandages. Burned victims can now place the bandage with the dried tissue fish skin over the burned area. The dried tissue fish skin will then transfer over the burned area and create a new layer of skin to heal. Currently a majority of the companies only work on Research and Development for wet tissues. Wet tissues for fish skin are capable of healing the burned skins, however, they cannot be implemented into bandages as only dried tissues can. Fish skin wet tissues also require a long period of time to rinse off chemical before safely apply on the burned patients. The cost of the dried tissue fish skin can be very economical, and that will provide affordable treatment for patients.

### BRIEF DESCRIPTION OF DRAWINGS

FIG. 1: [9] Illustrates the concept of creating Revi-Aid™

FIG. 1: [3] Illustrates the Tilapia fish skin that has been treated and conditioned at dried tissue stage.

FIG. 1: [1] Illustrates the adhesive strip with holes for air-breathe.

FIG. 1: [2] Illustrates the sterile gauze glued with the adhesive strip and holds the fish skin in place.

FIG. 2: [1] Illustrates the adhesive strip with holes for air-breathe

FIG. 2: [2] Illustrates the sterile gauze.

FIG. 2: [3] Illustrates the Tilapia fish skin at dried tissue stages and sterilized.

FIG. 2: [4] Illustrates the non-stick paper or plastic.

FIG. 3: [5] Illustrates the airtight vacuum pouch to keep the bandage from exposing to contaminated air.

FIG. 3: [6] Illustrates the guided tear line to open pouch and expose the bandage.

FIG. 3: [7] Illustrates the pulled tab to remove the plastic or non-stick paper to expose the dried tissue fish skin.

FIG. 3: [8] Illustrates the outer view of bandage cover by plastic or non-stick paper and adhesive strip.

FIG. 3: [9] Illustrates the Revi-Aid™ bandage.

FIG. 4: [1] Illustrates side view of the adhesive strip with holes for air-breathe.

FIG. 4: [2] Illustrates side view of the sterile gauze layer.

FIG. 4: [3] Illustrates side view of the Tilapia fish skin layer.

FIG. 4: [4] Illustrates side view of the plastic or non-stick paper to cover the fish skin and sterile gauze from exposure to air.

FIG. 5: [7] Illustrates top view of the pulled tab to remove the plastic or non-stick paper.

FIG. 5: [8] Illustrates outer view of bandage from top view.

FIG. 5: [9] Illustrates Revi-Aid™ bandage interior from top view.

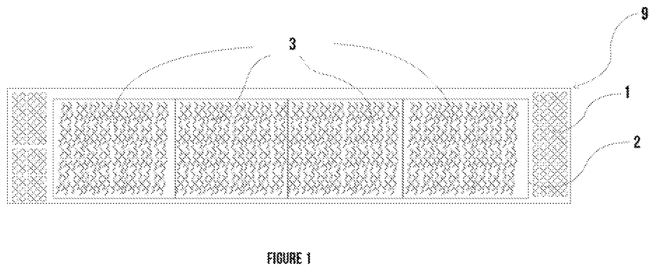

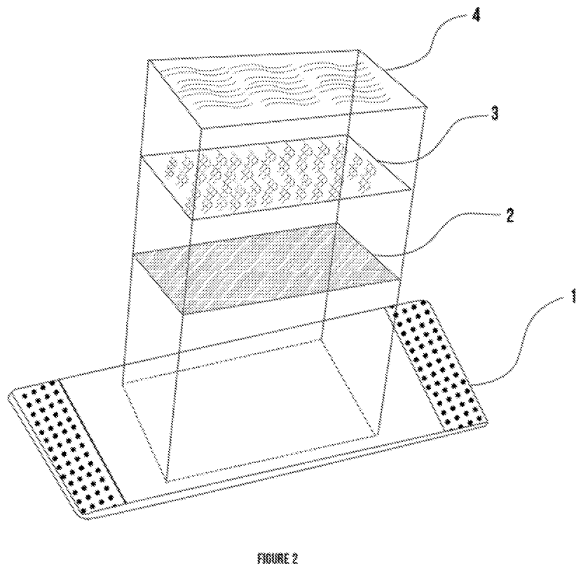

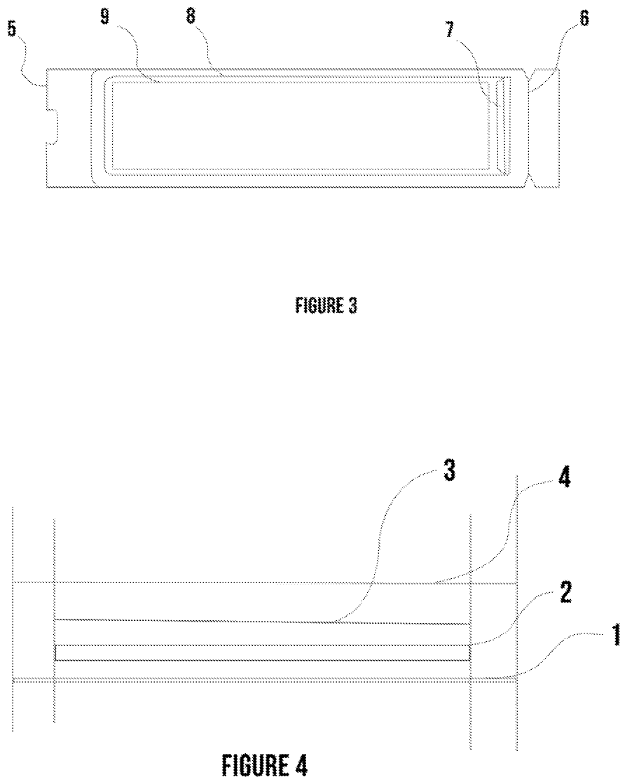

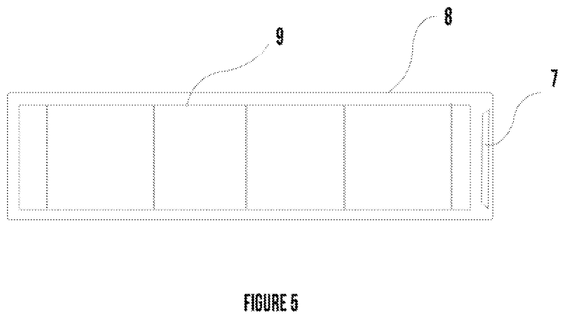

1. A new type of bandage called Revi-Aid™ contains sterile gauze and dried tissue fish skin. The dried tissue fish skin, specifically Tilapia, naturally contains collagen to accelerate the healing process of skin injuries or flesh wounds.

2. This type of bandage called Revi-Aid™ is created to intentionally cover the open wound for the burned patients; therefore, the size of Revi-Aid™ will be larger comparing to traditional medical dressing bandages.

3. The adhesive strip and the non-stick plastic coating paper, along with the sterile gauze layer, will protect the dried tissue fish skin from exposure to air. That will preserve the sterile stage and dried stage for the fish skin until it is removed and ready-to-use when applying the fish skin surface to the open wounds or skin injuries.
The purpose of the Revi-Aid™ will minimize the risk of open wounds or skin injuries from exposure to contaminated air, which may cause infections.
